(19) United States
(12) Patent Application Publication
(10) Pub. No.: US 2024/0416315 A1
(43) Pub. Date: Dec. 19, 2024

(54) CAPTURING OLIGONUCLEOTIDES IN SPATIAL TRANSCRIPTOMICS

(71) Applicant: 10X Genomics, Inc., Pleasanton, CA (US)

(72) Inventor: Felice Alessio Bava, Rome (IT)

(73) Assignee: 10X Genomics, Inc., Pleasanton, CA (US)

(21) Appl. No.: 18/819,856

(22) Filed: Aug. 29, 2024

Related U.S. Application Data
(63) Continuation of application No. 17/161,007, filed on Jan. 28, 2021, now Pat. No. 12,076,701.
(60) Provisional application No. 62/985,281, filed on Mar. 4, 2020.
(60) Provisional application No. 62/968,312, filed on Jan. 31, 2020.

(51) Int. Cl. B01J 19/00

(52) U.S. Cl. CPC B01J 19/0046; B01J 2219/00608; B01J 2219/00722

(57) ABSTRACT

Disclosed are extended and/or branched oligonucleotide capture probe assemblies for use in spatial transcriptomics systems, and methods for making the capture probe assemblies.

### RELATED APPLICATIONS

This application is a continuation of U.S. application Ser. No. 17/161,007, filed on Jan. 28, 2021, which claims priority to U.S. Provisional Application Ser. No. 62/985,281, filed on Mar. 4, 2020 and U.S. Provisional Application Ser. No. 62/968,312, filed Jan. 31, 2020. The entire contents of which are hereby incorporated by reference herein in their entireties.

### FIELD

This invention relates to improved capturing of biological analytes using spatial transcriptomics systems, especially when biological analytes are located remote from the surface of a biological tissue sample. The invention specifically relates to extended and/or branched capture probes that reach into a tissue sample and/or increase capture probe density within the tissue sample. The system and methods disclosed increase the analyte capturing potential of spatial transcriptomics arrays.

### BACKGROUND

The field of spatial transcriptomics encompasses systems and methods for obtaining spatially informative data on expression of analytes, typically mRNAs, from hundreds to thousands of individual cells in a tissue.

In some examples of these systems and methods, a tissue section is overlaid onto a planar, high-density array of oligonucleotides that are attached to a support. The oligonucleotides of the array encode spatial barcodes, which identify the spatial location of individual oligonucleotides on the support. The oligonucleotides also encode analyte-capture domains that can capture specific analytes released from the overlying cells (e.g., by hybridization of the oligonucleotide's analyte-capture domain to specific analyte mRNAs from the cells). The analyte capture domains are linked to a unique molecular identifier or UMI encoded in the oligonucleotides. The UMI uniquely identifies the captured analyte.

When cells of a tissue overlaid onto an oligonucleotide array are permeabilized, analytes are released from the cells and diffuse to the surface of the array, where the analytes contact the oligonucleotides of the array. When a migrating analyte contacts an analyte-capture domain that is specific for that analyte, the analyte is captured by that oligonucleotide. Nucleotide sequencing of oligonucleotides that have captured analytes identifies the analyte, the UMI, as well as the spatial barcode of the oligonucleotide. This information provides for assignment of specific captured analytes to specific spatial locations on the array where the analytes were captured.

Additionally, imaging of the original tissue section, and the array including fiducial markers onto which the tissue section was overlaid, provides for assignment of specific cells of the tissue to specific spatial locations on the array that directly underlie the cells. Image and sequencing correlation of the locations of individual cells on the array, with the analytes captured at those locations, yields a spatial map of analyte expression for the cells of the tissue.

Systems, reagents and methods for improving spatial transcriptomic interrogation of tissues are actively being developed.

### SUMMARY

Disclosed here are extended and/or branched assemblies of analyte capture probes for use in spatial transcriptomics systems. These capture probe assemblies extend, from the surface of an oligonucleotide array, into an overlaid tissue section. The branching nature of the capture probe assemblies increases the density of capture domains within a space in the tissue section. Because of their extended and branching properties, the capture probe assemblies capture more analytes (i.e., decrease the probability that an analyte will not be captured) and decrease the distance an analyte may have to migrate after release from a cell before it contacts a capture probe, as compared to conventional capture probe arrangements. The extended/branched capture probe assemblies, therefore, increase resolution of spatial transcriptomics systems. Disclosed are methods for making an assembly of oligonucleotide probes comprising: i) providing an oligonucleotide array that includes a support having a plurality of first probes affixed to the support, the plurality of first probes including a first nucleotide hybridization sequence; and ii) contacting the oligonucleotide array with: a) a plurality of second probes that include a second nucleotide hybridization sequence and a third nucleotide hybridization sequence, wherein the second nucleotide hybridization sequence is complementary to the first nucleotide hybridization sequence; and b) with a plurality of third probes that include a fourth nucleotide hybridization sequence complementary to the third nucleotide hybridization sequence; wherein the contacting is performed under conditions such that the first nucleotide hybridization sequence of the first probes is hybridized to the second nucleotide hybridization sequence of the second probes, and such that the third nucleotide hybridization sequence of the second probes is hybridized to the fourth nucleotide hybridization sequence of the third probes. Additionally, the contacting of the plurality of first probes, second probes and third probes may be done simultaneously; and wherein the method additionally includes: iii) first, hybridizing the plurality of first probes to the plurality of second probes at or below the first melting temperature; and iv) second, hybridizing the plurality of second probes to the plurality of third probes at or below the second melting temperature, but at a temperature greater than the first melting temperature.

Also disclosed are compositions comprising: i) an oligonucleotide array, including a support having a plurality of first probes affixed to the support, the plurality of first probes including a first nucleotide hybridization sequence; and ii) a plurality of second probes including a second hybridization sequence complementary to the first nucleotide hybridization sequence; wherein the first nucleotide hybridization sequence of the first probes is hybridized to the second nucleotide hybridization sequence of the second probes. Additionally, the plurality of second probes may include a third nucleotide hybridization sequence, and additionally comprising: iii) a plurality of third probes including a fourth nucleotide hybridization sequence complementary to the third nucleotide hybridization sequence; wherein the third nucleotide hybridization sequence of the second probes is hybridized to the fourth nucleotide hybridization sequence of the third probes In some examples, a melting temperature for a duplex formed by hybridization of the first nucleotide hybridization sequence with the second nucleotide hybridization sequence is greater than a melting temperature for a duplex formed by hybridization of the third nucleotide hybridization sequence with the fourth nucleotide hybridization sequence.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

The following U.S. patents and U.S. published patent applications are each incorporated by reference in their entirety into this application:
U.S. Pat. No. 9,593,365 (Ser. No. 14/434,274), issued Mar. 14, 2017 and titled, “Methods and Product for Optimising Localized or Spatial Detection of Gene expression in a Tissue Sample”;
U.S. Pat. No. 10,030,261 (Ser. No. 14/111,482), issued Jul. 24, 2018 and titled, “Method and Product for Localized or Spatial Detection of Nucleic Acid in a Tissue Sample”;
U.S. Pat. No. 10,774,374 (Ser. No. 15/565,637), published Jul. 4, 2019 and titled, “Spatially Distinguished, Multiplex Nucleic Acid Analysis of Biological Specimens;
U.S. Pat. No. 10,550,429 (Ser. No. 16/426,762), issued Feb. 4, 2020 and titled, “Methods and Systems for Processing Polynucleotides”; and
U.S. Pat. No. 10,590,244 (Ser. No. 16/178,430), issued Mar. 17, 2020 and titled, “Compositions, Methods, and Systems for Bead Formation Using Improved Polymers.”

Other references incorporated by reference may be listed throughout the application.

### BRIEF DESCRIPTION OF THE DRAWINGS

In the accompanying drawings, which are incorporated in and constitute a part of the specification, embodiments of the disclosed inventions are illustrated. It will be appreciated that the embodiments illustrated in the drawings are shown for purposes of illustration and not for limitation. It will be appreciated that changes, modifications and deviations from the embodiments illustrated in the drawings may be made without departing from the spirit and scope of the invention, as disclosed below.

FIG. 1 is a schematic drawing (side view) illustrating an example of a spatial array oligonucleotide capture probe.

FIG. 2 is a schematic drawing (top view) that illustrates an example of a region (e.g., an array spot) on a support that contains a species of capture probe (i.e., a plurality of a species of capture probes).

FIG. 3 is a schematic drawing (top, angular view) that illustrates an example of a region on a support with multiple species of capture probes (e.g., a spatial array).

FIG. 4 is a schematic drawing (side view) that illustrates an example of a capture probe attached to a support.

FIG. 5 is a schematic drawing illustrating an embodiment of a scaffolding arrangement of oligonucleotide capture probes.

FIG. 6 is a schematic drawing illustrating another embodiment of a scaffolding arrangement of oligonucleotide capture probes.

FIG. 7 illustrates a schematic of an example of a branched arrangement of assembled oligonucleotide capture probes.

FIG. 8 illustrates a schematic of an example of a scaffolding arrangement of oligonucleotide capture probes.

### DETAILED DESCRIPTION

Spatial arrays include oligonucleotide capture probes attached to a support. When a tissue section is overlaid onto the surface of an array and analytes are released from cells of the tissue, analytes from the cells migrate away from the cells, contact the capture probes of the spatial arrays and are bound by domains in the capture probes, called capture domains, that are specific for given analytes. However, capture probes may favor capture of analytes proximal to the surface of the tissue section in contact with the spatial array and may less efficiently capture analytes from cells located deeper within the tissue section. In some examples, capture probes may not extend far enough into the depth of the overlying three-dimensional tissue structure (e.g., 7-15 μm thickness) to contact analytes from cells located there. Even for analytes from cells that are proximal to the surface of the spatial array, the density of capture probes may not be high enough to prevent existence of “dead areas” in which there are no capture probes, or where there are not enough capture probes, to capture the available analytes. Consequently, analytes may not be captured or, when analytes are captured, they may be captured by capture probes that are distant from the cells that released the analytes. Capture of an analyte by a non-adjacent capture probe may limit the ability to map the analyte back to the location of the cell in the tissue section that actually released it and may degrade resolution of the spatial system.

The present disclosure provides systems and techniques for self-assembling multiple oligonucleotide probes into extended and/or branched scaffolds. The scaffolds extend the reach of capture probes to points distal from the surface of the spatial arrays and into the depth of the tissue section overlying a spatial array. Branching of the scaffolds increases the density of capture probes, prevents dead areas that contain no capture probes and lessens the probability that an analyte will not be captured. The branched scaffolds also increase resolution of the spatial array systems by decreasing the distance that an analyte has to migrate when released from a cell, before the analyte contacts and is captured by an oligonucleotide capture probe.

### Definitions

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

Herein, “amplification product” refers to molecules that result from reproduction or copying of another molecule. Generally, the molecules copied or reproduced are nucleic acid molecules, specifically DNA or RNA molecules. In some examples, the molecule reproduced or copied may be used as a template for the produced molecules. In some examples, an analyte captured by the capture domain of an oligonucleotide may be used as a template to produce an amplification product. In some examples, an mRNA captured by the capture domain of an oligonucleotide may be used as a template to produce a cDNA amplification product. Various enzymes (e.g., reverse transcriptase) may be used for this process. The cDNA amplification product may in turn act as a template for amplification that may also be called amplification products. Various enzymes (e.g., Taq polymerase) may be used for this process.

Herein, “analyte” refers to a substance whose chemical constituents are being identified and/or measured. Generally, this application refers to analytes from and/or produced by cells. Any or all molecules or substance from or produced by a cell may be referred to herein as analytes. Chemically, cellular analytes may include proteins, polypeptides, peptides, saccharides, polysaccharides, lipids, nucleic acids, and other biomolecules.

Herein, “array” refers to a region on a support that contains multiple demarcated regions of oligonucleotides, interspersed with intervening regions that do not contain oligonucleotides. In some examples, these regions may be referred to as “oligonucleotide arrays” or “capture areas”. The arrays herein generally have oligonucleotides that contain spatial barcodes and, thus, the arrays may be referred to as “spatial” arrays.

Herein, “associated with” generally describes a connection or relationship between two things. Herein, “associated with” may be used to refer to the results of one or more of at least three processes. The first process is cell segmentation, where individual cells overlaid onto an array are matched with or assigned to one or more array spots that are directly underneath the overlaid cells. The second process is assignment of analytes (e.g., nucleotide sequences), generally representing specific mRNAs from cells overlaid onto an array, to array spots that capture the specific mRNA, using spot-specific barcodes encoded by the oligonucleotides of the array. The third process is assignment of nucleotide sequences, representing specific mRNAs from cells overlaid onto an array, to specific cells overlaid onto the array.

Herein, “assembly,” when used as a noun, refers to the layered configurations of oligonucleotide capture probes described herein. An assembled arrangement of capture probes may also be referred to as a scaffold. When used as a verb, “assembly” may refer to the process of building such a configuration of oligonucleotide capture probes.

Herein, “barcode,” generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). Barcodes can have a variety of different formats. For example, barcodes can include polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads. In some examples, a barcode may be a nucleotide sequence that is encoded by, linked to or associated with one or more oligonucleotides. In some examples, a specific barcode may correlate with a location of a barcode, on a support, for example. A barcode used to convey locational information may be called a spatial barcode.

Herein, “barcoded molecule” or, in some examples, “barcoded nucleic acid molecule” generally refers to a molecule or a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence (e.g., targeted by a primer sequence) or a non-targeted sequence. For example, in the methods, systems and kits described herein, hybridization and reverse transcription of the nucleic acid molecule (e.g., a messenger RNA (mRNA) molecule) of a cell with a nucleic acid barcode molecule (e.g., a nucleic acid barcode molecule containing a barcode sequence and a nucleic acid primer sequence complementary to a nucleic acid sequence of the mRNA molecule) results in a barcoded nucleic acid molecule that has a sequence corresponding to the nucleic acid sequence of the mRNA and the barcode sequence (or a reverse complement thereof). A barcoded nucleic acid molecule may be a nucleic acid product. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the mRNA as well as the sequence of the spatial barcode thereby determining the locational position of the mRNA along with its identity. Herein, molecules stated to have a “common barcode sequence” refers to molecules that are labeled or identified with the same barcode sequence.

Herein, “base-paired” generally refers to the situation where two complementary nucleic acids have formed hydrogen bonds between complementary nucleotides in the different strands. Two such nucleic acid strands may be referred to as hybridized to one another.

Herein, “branched” generally refers to a particular arrangement of oligonucleotide capture probes within an assembly that increases the density of capture domains in a space.

Herein, “capable” means having the ability or quality to do something.

Herein, “capture” generally refers to the capability of a first substance to interact with and/or bind a second substance where, for example, the second substance is part of a population of other substances. An analyte may be captured. In some examples, capture refers to identification of a target nucleic acid molecule (e.g., an RNA) by its hybridization to a capture probe, and/or amplification of a target nucleic acid molecule or a nucleic acid probe hybridized to it (e.g., an RNA or a probe hybridized to the RNA) using, for example polymerase chain reaction (PCR) and/or nucleic acid extension of a target nucleic acid molecule or a capture probe hybridized to it using, for example reverse transcription reactions.

Herein, “capture probe” refers to a molecule (e.g., an oligonucleotide) that contains a capture domain.

Herein, “capture domain” means a part of a molecule that is capable of binding or capturing a substance. An analyte capture domain may be capable of capturing analytes that may include proteins, polypeptides, peptides, saccharides, polysaccharides, lipids, nucleic acids, and other biomolecules. In some examples, the analyte capture domain may be a nucleotide sequence capable of hybridizing to an analyte that contains a complementary nucleotide sequence. Herein, “nucleotide capture sequence” refers to a first nucleotide sequence that is capable of capturing (e.g., by hybridizing to) a second nucleotide sequence. In some examples, an analyte capture domain may contain modified nucleotides.

Herein, “cell block” refers to cells embedded in a medium. In some examples, the medium may be a gel, like agarose, hydrogel, polyacrylamide, OCT, and the like.

Herein, “complementary,” in the context of one sequence of nucleic acids being complementary to another sequence, refers to the ability of two strands of single-stranded nucleic acids to form hydrogen bonds between the two strands, along their length. A complementary strand of nucleic acids is generally made using another nucleic acid strand as a template. A first nucleotide that is capable of hybridizing to a second nucleotide sequence may be said to be a complement of the second nucleotide sequence.

Herein, “configured to” generally refers to a component of a system that can perform a certain function.

Herein, “contact” refers to physical touching of separate substances or objects. “Contacting” refers to causing separate substances to physically touch one another.

Herein, “cryosection” refers to a section of a tissue or a cell block for visual or microscopic examination made from a fresh frozen sample by a cryostat. A cryosection is a type of “section” as defined herein.

Herein, “diffusion” refers to travel of an analyte released from a cell that is overlaid onto a spatial array, to capture probes that are not the capture probes nearest to the overlaid cell. See definition of “migration” for more detail.

Herein, “discrete” means separate or individual.

Herein, “duplex” refers to a double-strand nucleic acid. Herein, duplexes are generally formed between complementary hybridizing nucleotide sequences.

Herein, “extended” generally refers to a particular arrangement of oligonucleotide capture probes within an assembly that increase the length or reach of capture domains in a space.

Herein, “fix,” refers to formation of covalent bonds, such as crosslinks, between biomolecules or within molecules. The process of fixing cells for example, is called “fixation.” The agent that causes fixation is generally referred to as a “fixative” or “fixing agent.” “Fixed cells” or “fixed tissues” refers to cells or tissues that have been in contact with a fixative under conditions sufficient to allow or result in formation of intra- and inter-molecular crosslinks between biomolecules in the biological sample. Fixation may be reversed and the process of reversing fixation may be referred to as “un-fixing” or “decrosslinking.” Unfixing or decrosslinking refers to breaking or reversing the formation of covalent bonds in biomolecules formed by fixatives. Non limiting examples of fixatives or fixing agents include methanol, paraformaldehyde, formalin, and acetone to name a few.

Herein, “generate” means to make or produce. Generally, herein, generate is used to describe producing complementary nucleic acid molecules (e.g., making an amplification product) using a template nucleic acid molecule.

Herein, “hybridize” refers to a nucleotide sequence of a single-stranded nucleic acid molecule forming a complex with a nucleic acid molecule having a complementary nucleotide sequence. Generally, the complex forms through hydrogen bonding between complementary nucleotide bases in separate nucleic acid molecules.

Herein, “hybridizing nucleotide sequence” refers to a nucleotide sequence, within an oligonucleotide for example, that is capable of hybridizing with a complementary nucleotide sequence in a target nucleic acid molecule present on or within a cell from a tissue sample (e.g., cellular RNA). When a hybridizing nucleotide sequence is of such a length that it hybridizes with a complementary, either fully or partially, nucleotide sequence that is unique to a target nucleic acid molecule(s) (e.g., cellular RNA or family of RNAs), the hybridizing nucleotide sequence may be said to hybridize to the same target nucleic acid molecule (e.g., the same RNA).

Herein, “immobilize” means to restrict or prevent movement.

Herein, “in common” means a property, characteristic, feature, etc., that is possessed by separate things. Herein, “in common” may be used, for example, to refer to a nucleotide sequence that is possessed or encoded by two or more separate molecules.

Herein, “intermediate agent” refers to an identifier of a particular mRNA that is not itself the mRNA. In some examples, an intermediate agent of a particular mRNA may be a DNA sequence complementary to the mRNA (i.e., cDNA) or a DNA sequence complementary to the cDNA.

Herein, “intervening region” or “interspot space” refers to areas on a support of an array that do not contain attached oligonucleotides. Herein, “library” refers to a collection of molecules having nucleotide sequences that are generally representative (e.g., comprising the same nucleotide sequences or complementary nucleotide sequences) of nucleotide sequences present in the molecules from the target nucleic acids. Generally, the molecules from which a library is made act as templates for synthesis of the collection of molecules that make up the library. The “library” may be, or may be produced from, amplification products of the target nucleic acid. Herein, libraries can be created from amplification of a mRNA analyte, or copies thereof, captured on an array. Therefore, the library can be derived from the captured target nucleic acid.

Herein, “melting temperature” or “Tₘ” of a nucleic acid hybrid (i.e., two nucleic acid stands hybridized through hydrogen bonding) refers to a temperature at which hybridized nucleic acid strands become single-stranded. As known in the art, Tm may depend on the length of hybridizing nucleic acid sequences (i.e., the number nucleotides in the duplex), the base-pair composition of the duplex, as well as other factors. Generally, it is possible to design a duplex (e.g., see definition for “hybridizing nucleotide sequence”) having a specific Tₘ, and/or to design a series of duplexes having a progression of Tₘ's (e.g., three duplexes where the first has a relatively low Tₘ, the second has an intermediate Tₘ, and the third has a relatively high Tₘ).

Herein, “migration” refers to movement of an analyte from a cell that is overlaid onto a spatial array, to a capture probe that is attached to the surface of the array. In some examples of migration, analytes released from cells on an array may migrate in such a way that they contact capture probes located directly under the cell and/or are in contact with the cell. In other examples of migration, analytes released from cells on the array may migrate in such a way that they do not contact capture probes nearest the cell that released the analyte. Instead, at least some of the analytes released from a cell may travel away from the cells (e.g., laterally, orthogonally, etc.) and contact capture probes that are not nearest the cell that released the analyte. In some examples, the analytes may contact capture probes adjacent, or even not adjacent, to capture probes that lie underneath or contact the cells overlaid onto an array. Herein, travel of analytes to capture probes that are not those nearest to an overlaid cell is referred to as “diffusion.” Diffusion is a type of migration. In the present disclosure, migration and diffusion are passive, unless otherwise stated.

Herein, “oligonucleotide” means a linear polymer of nucleotides, in some examples 2′-deoxyribonucleotides. Oligonucleotides are single stranded. Oligonucleotides can be of various lengths. Oligonucleotides can include modified nucleotides as known in the art.

Herein, “origin” refers to the source of something. Something that is stated to have a specific origin may be said to “originate” from that source.

Herein, “permeable” refers to something that allows certain materials to pass through it. “Permeable” may be used to describe a cell in which analytes in the cell can leave the cell. “Permeabilize” is an action taken to cause, for example, a cell to release its analytes. In some examples, permeabilization of a cell is accomplished by affecting the integrity of a cell membrane such as by application of a protease or other enzyme capable of disturbing a cell membrane allowing analytes to diffuse out of the cell.

Herein, “primer” means a single-stranded nucleic acid sequence that provides a starting point for DNA synthesis. Generally, a primer has a nucleotide sequence that is complementary to a template, and has an available 3′-hydroxyl group to which a transcriptase or polymerase can add additional nucleotides complementary to corresponding nucleotides in the template, to synthesize a nucleic acid strand in the 3′ to 5′ direction.

Herein, “print” means to apply something to a substrate or surface in a particular way or to a particular location. Printing refers to the act of applying something to a substrate.

Herein, “resolution” is generally used to describe the ability of a spatial analysis system to attribute, correlate or match expression of an analyte to one or more cells. High resolution is desirable and refers to the situation where expression of analytes can be ascribed to single cells.

Herein, “RNA capturing probe” refers to a nucleic acid molecule capable of hybridizing to an RNA.

Herein, “sample” or “biological sample” generally refers to a collection of cells or to a tissue. Generally, a tissue contains multiple cells, often similar cells that may perform the same or similar functions. The sample may be a cell sample. The sample may be a cell line or cell culture sample. The sample can include one or more cells, or one or more cell aggregates or clusters. The biological sample may be derived from another sample. The sample may be a tissue sample, such as a biopsy, core biopsy, needle aspirate, or fine needle aspirate. Example tissue types in animals may include connective, epithelial, brain, adipose, muscle and nervous tissue. The sample may be a fluid sample, such as a blood sample, urine sample, or saliva sample. The sample may be a skin sample. The sample may be a cheek swab. The sample may be a plasma or serum sample. In some examples, a sample may comprise any number of macromolecules, for example, cellular macromolecules or cellular analytes. The present disclosure is not limited to any particular type of tissue.

Herein, “section” generally refers to a thin layer or slice from a larger object.

Herein, “spatial” refers to a location within or on a space. In some examples, the space may be a two-dimensional space.

Herein, “species” generally refers to multiple oligonucleotides that have something in common. Generally, oligonucleotides considered to be part of the same species have at least one barcode in common. In some examples, the common barcode may be associated with a particular or a group of capture domain(s). In some examples, the common barcode may be associated with oligonucleotides on a support, for example, that are in proximity to one another. In some examples, the common barcode may be encoded by the oligonucleotides that are part of an array spot.

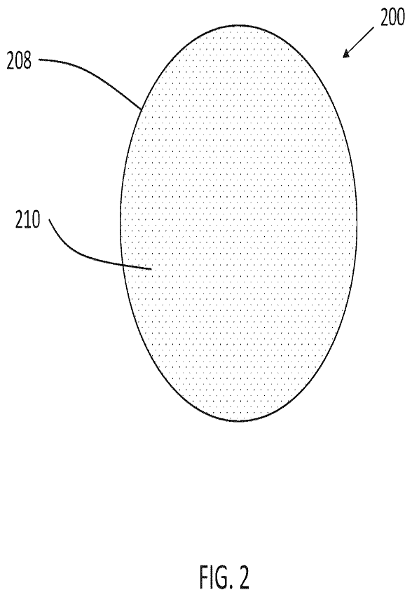

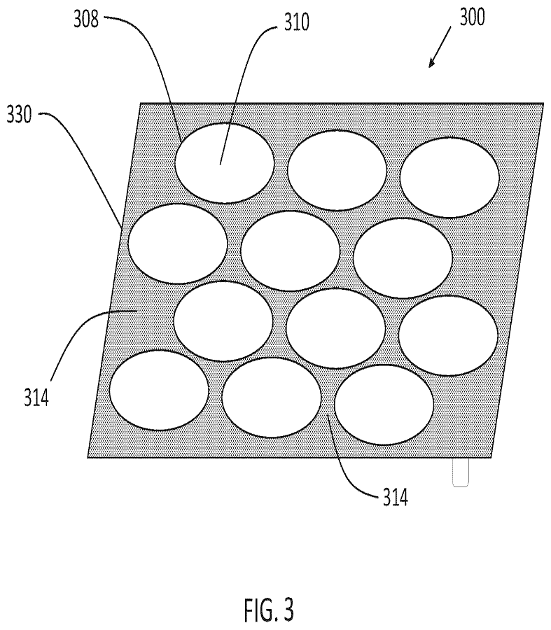

Herein, “spot” or “array spot” refers to an area on a support that contains a generally uninterrupted area of oligonucleotides attached to the support. Spots can also be referred to as “features”. Generally, these areas have boundaries, beyond which, there may not be oligonucleotides attached to the support. An example array spot is shown as 208 in FIG. 2. In FIG. 3, multiple array spots 308 are shown on a support 330.

Herein, “support,” when used as a noun, refers to something that serves, for example, as a foundation for another thing. In some examples, the support may be larger, more easily worked with, or more easily tracked or visualized than the thing being supported. A support may be a solid support. In some instances, a support may be dissolvable, disruptable, and/or degradable. In some cases, a support may not be degradable. A support may comprise glass, plastic, metal, and/or other substances. In some cases, the support can be rigid. In other cases, the support may be flexible and/or compressible. In some examples, a support may be referred to as a “substrate.”

Herein, “surface” means the outside part or upper layer of something. Herein, a “surface” of an array generally refers to a surface of a support or substrate that has oligonucleotides attached.

Herein, “template” refers to one single-stranded nucleic acid acting as a “template” for synthesis of another complementary single-stranded nucleic acid. For example, RNA can act as a template for synthesis of a complementary DNA strand synthesized using reverse transcriptase. A single-stranded DNA can act as a template for synthesis of a complementary DNA strand, most often by a DNA polymerase.

Herein, “unique” means one of a kind or unlike something else. In some examples, a “unique” mRNA may refer to an mRNA encoded by a single-copy gene.

Herein, “unique molecular identifier” or “UMI” generally refers to an identifier of a particular analyte captured by a capture probe.

### Obtaining Spatially Aligned Analyte Expression Data From Cells and Tissues

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample, including a mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample, it serves as a proxy for the analyte.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10 × Genomics Support Documentation website, and can be used herein in any combination. The above references, if US Patents or US Patent Publications, are incorporated herein by reference in their entirety. Further non-limiting aspects of spatial analysis methodologies and compositions are described herein.

Array-based spatial analysis methods involve the transfer of one or more analytes from a biological sample to an array of features (e.g., spots) on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent, including a ligation product or an analyte capture agent, or a portion thereof), or derivatives thereof (see, e.g., Section (II)(b)(vii) of WO 2020/176788 and/or U.S. Patent Application Publication No. 2020/0277663 regarding extended capture probes; incorporated herein by reference in their entirety). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” (as opposed to an extended assembly of capture probes, as described herein) refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II) (a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up-and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

In some cases, spatial analysis can be performed by detecting multiple oligonucleotides that hybridize to an analyte. In some instances, for example, spatial analysis can be performed using RNA-templated ligation (RTL). Methods of RTL have been described previously (See, e.g., Credle et al., Nucleic Acids Res. 2017 Aug. 21; 45(14):e128). Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a two dimensional or three-dimensional map of the analyte presence and/or level.

In some cases, a map of analyte presence and/or level can be aligned to an image of a biological sample using one or more fiducial markers (e.g., objects placed in the field of view of an imaging system which appear in the image produced, as described in the Substrate Attributes Section and Control Slide for Imaging Section of WO 2020/123320). Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

Systems and methodologies in the field of spatial transcriptomics are designed to obtain spatially resolved analyte expression data (e.g., genomics, proteomics, transcriptomics) from tissues. In some examples, a tissue may be overlaid onto a support comprising barcoded oligonucleotides or capture probes. Generally, the oligonucleotides comprise a spatial barcode, which is correlated with and is an identifier for the location of the particular oligonucleotide on the support (e.g., in some examples, oligonucleotides having known barcode sequences are printed onto designated areas of the support). When analytes are released from a biological sample and migrate toward and contact the barcoded oligonucleotides, the barcoded oligonucleotides capture, or hybridize to, the analytes. In some examples, mRNAs may be the analytes and barcoded oligonucleotides may capture mRNAs having specific nucleotide sequences by hybridization, for example the barcoded oligonucleotides comprise a poly(T) capture domain that can hybridize a poly(A) tail of a mRNA. In the examples where mRNA is the analyte, reverse transcription of the captured mRNA can be initiated using added primers, and cDNA is produced using the barcoded oligonucleotide as a template. The resultant cDNA that is synthesized incorporates the barcodes included in the barcoded oligonucleotide or capture probe. The cDNAs may be amplified. A library of the cDNAs/amplified cDNAs is prepared and nucleotide sequences of the libraries are obtained. Nucleotide sequences of the spatial barcodes provides for the data for an mRNA transcript to be mapped back to its location on the support, and by also obtaining an image of the tissue and cells overlaid onto the support at the beginning of the procedure, mRNA transcripts may be mapped to the location in the overlaid tissue, where the mRNA was expressed.

In some examples, a planar support on the surface of which is attached a spatially ordered arrangement of barcoded oligonucleotides comprising analyte capture domains is used. In some examples, an analyte capture domain may be an oligo(dT) sequence for capturing poly(A) sequences of eukaryotic mRNA. Other sequences may be used to capture specific nucleic acids, including specific mRNAs. The arrangement of the oligonucleotides on the surface of the support can be known because the oligonucleotides comprise spatial barcodes. In some examples, the oligonucleotides, with known spatial barcodes, are printed in a known pattern onto specific, known areas of the surface of the planar support in a predetermined arrangement. A tissue is then applied to the surface of the support and analytes (e.g., mRNA) are released from the cells that make up the tissue. mRNAs released from the tissue migrate to the surface of the support and hybridize to oligo(dT) capture domain sequences of the attached oligonucleotides. The hybridized mRNAs are amplified using reverse transcription into complementary oligonucleotides that include sequences from the captured mRNA linked to the spatial barcode of the oligonucleotide to which the mRNA bound. Obtaining and decoding the nucleotide sequences of the complementary oligonucleotides reveals where on the support specific mRNAs bound to oligonucleotides. These locations are then correlated to regions of the tissue that was applied to the surface of the support.

In modifications of the above method, a tissue sample may be probed for expression of specific proteins using antibodies. The antibodies may have attached nucleotide tags having a specific nucleotide sequence that capture domains of the barcoded molecules on a support are designed to capture through hybridization. Thus, proteomic data can be obtained from the oligonucleotide arrays.

In modifications of the above method, a tissue sample may be probed for presence or absence of genetic mutations, variants, diversity, polymorphisms and the like in genomes, including single-nucleotide polymorphisms (SNPs) or single-nucleotide variants (SNVs) in genomes of cells making up the tissue. In some examples, a probe for a SNP or SNV may include a specific nucleotide sequence that can differentially hybridize to a genomic sequence dependent on whether a SNP or SNV is present. In some examples, a probe for a SNP or SNV may include a nucleotide sequence that can hybridize to a genomic sequence that is linked to (e.g., upstream of downstream of) a genomic region that might contain the SNP or SNV. Extension of the hybridized sequence, using the region of the genome that might contain the SNP/SNV as a template, and nucleotide sequencing of the extension product, may be used to determine if the SNP/SNV is present in the extension product. In some examples, probes for specific SNPS or SNVs may be part of the capture domain of certain oligonucleotides that make up the oligonucleotide array. Other techniques may be used to detect SNPs and/or SNVs.

In modifications of the above method, a tissue sample may be probed for isoforms of genes, transcripts (e.g., alternative transcription start sites, alternatively spliced mRNAs) or proteins. In some examples, a probe for an isoform of a gene or transcript may be designed to hybridize to one form but not the other, or may be designed to hybridize to or near a region that may contain the isoform such that amplification and/or extension of the hybridized probe, and optional nucleotide sequencing of the amplified product, can detect presence or absence of specific isoforms. In some examples, a probe for an isoform of a protein may be an antibody designed to differentially bind to the different isoforms. The antibodies used may have attached nucleotide tags that can capture domains of the barcoded molecules on a support, as described above.

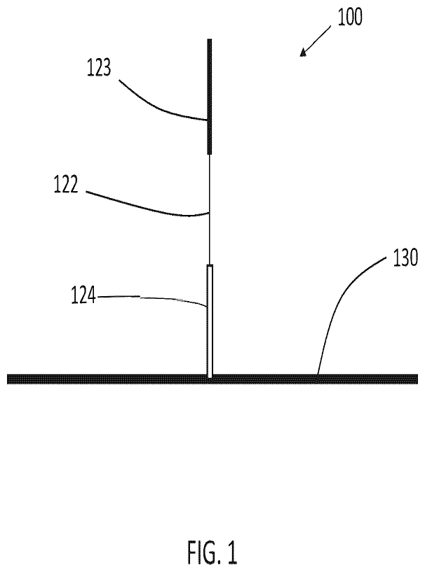

Related to the invention disclosed herein, FIG. 1 is a schematic drawing (side view) that illustrates an example of a barcoded oligonucleotide 100. The oligonucleotide 100 is shown attached to a support or substrate 130. The barcode molecule 100 may have a variety of regions. One example region is an analyte capture domain 123. Another example region is a barcode nucleotide sequence 122. The barcode sequence 122 may be common to a plurality of the oligonucleotides 100. A barcode sequence 122 may correspond to a location on the support 130 where the oligonucleotide 100 is attached or immobilized (e.g., spatial barcode). A unique molecular identifier (UMI) may also be included as part of the barcoded nucleotide oligonucleotide. A UMI sequence may correspond to a unique molecular identifier (UMI) associated with the oligonucleotide 100. The oligonucleotide 100 may have multiple barcode sequences 122. In some examples, an oligonucleotide may have a barcode sequence that corresponds to the oligonucleotide and a barcode sequence that corresponds to the location on the support where the oligonucleotide is attached. The oligonucleotide may have other or additional regions 124 (e.g., PCR handles, cleavage domains, sequencing primer domains, etc.).

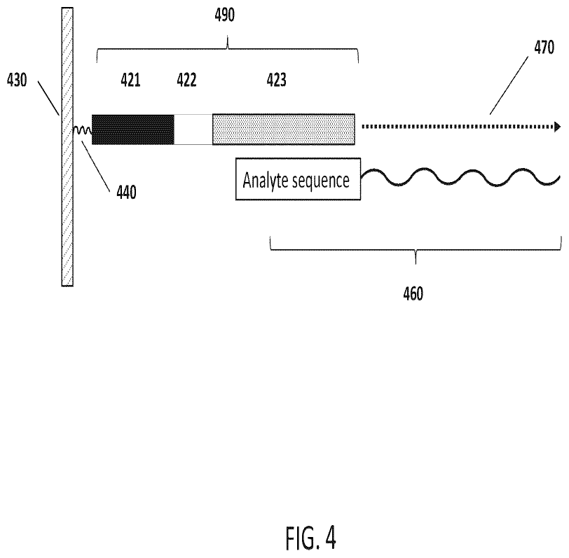

In some examples, referring to FIG. 4, a barcoded oligonucleotide or capture probe 490 comprises a barcode sequence 422 corresponding to a location on a support 430 to which the oligonucleotide is attached (as illustrated in this example, the oligonucleotide 490 is attached to the support 430 via a modification or chemical moiety 440 capable of attaching to the support 430). The modification or chemical moiety 440 attached to the support can be a cleavable moiety (e.g., photocleavable, chemically cleavable, enzymatically cleavable, etc.). The illustrated oligonucleotide 490 also comprises a sequence 423 (i.e., an analyte capture sequence or capture domain) complementary to a sequence of an analyte (e.g., mRNA molecule) 460 from a cell. In some instances, sequence 423 comprises a sequence specific for an mRNA molecule. In some instances, sequence 423 comprises a poly(dT) sequence. In some instances, sequence 423 comprises a defined nucleotide sequence, a semi-random nucleotide sequence or a random nucleotide sequence. Sequence 423 is hybridized to mRNA molecule 460 (i.e., the mRNA is captured by the 423 sequence) and extended via a nucleic acid reaction (e.g., a cDNA molecule 470 is generated in a reverse transcription reaction) generating a complementary oligonucleotide comprising barcode sequence 422 (e.g., a spatial barcode sequence, or a reverse complement thereof) and a sequence of the extended nucleic acid (e.g., cDNA 470) (or a portion thereof). A functional sequence 421, such as a primer binding site for amplification and/or a sequencing related primer binding site (e.g., a sequence used for a sequencing reaction), etc. is also included in the barcoded oligonucleotide or capture probe. In some examples, barcoded nucleic acid molecules can then be optionally processed as described elsewhere herein, e.g., to amplify the molecules and/or append sequencing platform specific sequences to the fragments. Barcoded nucleic acid molecules, or derivatives generated therefrom, can then be sequenced on a suitable sequencing platform. Nucleic acid barcode molecule 490 may be attached to support 430 optionally via a releasable linkage 440 (e.g., comprising a labile bond), such as those described in WO2020/047007A2 (Appl. No. PCT/US2019/048430), WO2020/047010A2 (Appl. No. PCT/US2019/048434), WO2020/047004A3 (Appl. No. PCT/US2019/048427), and WO2020/047005A1 (PCT/US2019/048428), each of which are incorporated by reference herein in their entirety.

FIG. 2 is a schematic drawing (top view) that illustrates an example region 200 of a support that contains a plurality of oligonucleotides, individual of the oligonucleotides shown in FIG. 1 and FIG. 4. Such a region, that contains an uninterrupted area of oligonucleotides attached to a support, may be referred to as a “spot,” “array spot”, “feature”, “partition” or “demarcated region” 208. In some examples, the oligonucleotides that make up a spot are printed onto the support. In the example, each dot 210 within the demarcated region 208 represents one or a plurality of oligonucleotides attached to the support. In some examples, the oligonucleotides 210 within the demarcated region 208 may all contain the same barcode sequence that corresponds to the location on the support where the oligonucleotides are attached (e.g., spatial barcode). In some examples, the analyte capture domains may be poly(dT). In some examples, the oligonucleotides 210 within the demarcated region 208 may contain different unique molecular identifiers. In some examples, the oligonucleotides 210 within the demarcated region 208 may contain different barcode sequences that correspond to the analyte capture domain encoded by the oligonucleotide (e.g., the oligonucleotides within the demarcated region 208 may have different analyte capture domains). In some examples, the oligonucleotides 210 within the demarcated region 208 may be said to represent a species of oligonucleotides. In some examples, a species of oligonucleotides may be oligonucleotides with at least one barcode nucleotide sequence in common. In some examples, the barcode sequence in common may be a barcode sequence corresponding to a location on a support to which the oligonucleotides are attached (e.g., spatial barcode). As described below, a support may contain multiple, adjacent demarcated regions 208. In some examples, the oligonucleotides of a region may all have the same spatial barcode. In some examples, the oligonucleotides of different regions may have different spatial barcodes. In some examples, the oligonucleotides of a single region may have originated from a single oligonucleotide that was bound to a flow cell, as is described below. In some examples, the single oligonucleotide may have been amplified using “bridge-amplification” on a flow cell. In some examples of the invention disclosed herein, a single demarcated region 208, may contain at least 500, at least 1000, at least 1500, at least 2000, at least 2500, at least 3000, at least 3500, at least 4000, at least 4500, at least 5000, at least 10,000, at least 100,000, at least 250,000, at least 500,000, at least 1,000,000 at least 1,500,000, at least 2,000,000 template oligonucleotides within a distance of 0.5, 1, 5, 10, 50, 100 or 500 μm of one another on the support.

FIG. 3 is a schematic drawing (top, angular view) of a larger area of a support than illustrated in FIG. 2. FIG. 3 illustrates an example of a region of a support containing multiple of the regions 200 shown in FIG. 2. FIG. 3 may be said to illustrate an array 300 which, herein, is a region on a support 330 that contains multiple of the spots 308 that contain multiple oligonucleotides 310 attached to the support 330. Each spot region contains multiple oligonucleotides 210, as discussed for FIG. 2. The array 300 may have areas on the support to which oligonucleotides 310 are not attached. Such areas may be called intervening regions 314. Some example arrays 300 may not have intervening regions 314 and, thus, may have continuous uninterrupted areas of oligonucleotides 310 attached to the support 314. Generally, the array 300 has multiple spots 308 of oligonucleotides 310 attached to a support 330, where the spot regions 308 are interspersed with intervening regions 314.

In some examples, the oligonucleotides 310 within a single spot have at least one barcode nucleotide sequence in common (e.g., spatial barcode). In some examples, a barcode sequence in common may correspond to a location on the support 330 to which the oligonucleotides 310 are attached. In some examples, the barcode sequence corresponding to a location on the support 330 may be different for oligonucleotides 310 within different spot regions 308. In some examples, a support 330 may have at least 50, 100, 500, 1,000, 5,000, 10,000, 20,000, 30,000, 40,000 50,000, 100,000, 500,000 or 1,000,000 spot regions 308 within one mm² on the support 330.

As described, analytes released from cells may be captured by capture domains of barcoded oligonucleotides attached to the support of the array. In some examples, cells that are overlaid onto an array may be between about 10-20 μm in diameter. In some examples, a spot 308 as illustrated in FIG. 3 may be at least 1, 2, 3, 4, 5, 6, 7, 8, 9, 10, 20, 30, 40, 50, 60, 70 or 80 μm in diameter. In some examples, a cell overlaid onto such an array may be entirely within the area of a single spot or may extend beyond the area of a single spot. In some examples, a tissue overlaid onto an array that is not entirely within the area of a single spot may be overlaid onto or cover, for example, 2, 3, 4, 5, 6, 7, 8, 9 or more different adjacent spots on the surface of the array. In some embodiments, cells and tissues on a spot may represent all one cell type, or different cell types.

Cells or tissues on an array can be permeabilized, wherein analytes (e.g., mRNAs) from the cells are released from the cells, migrate to the surface of the array and are captured by specific capture domains of the barcoded oligonucleotides. In some examples, the analytes may migrate and contact spots of the array over which the cells directly lie or contact. In some examples, the analytes may not migrate and contact spots that directly underlie or that contact the cells from which the analyte originates. Instead, analytes may diffuse away from the spot most proximal to the cell that released the analyte. For example, an analyte originating and released from a cell may migrate to and contact an array spot which it directly overlays or contacts. Additionally, or alternatively, an analyte originating and released from a cell may contact one or more adjacent or even non-adjacent array spots which are not under the cell proper. As such, an analyte may diffuse away from the spot that is directly under the cell from which it originates, wherein that analyte is captured by a barcoded oligonucleotide that is not necessarily indicative of its spatial location from its original cell.

In some examples of diffusion of analytes away from the array spots that are most proximal to cells that released the analytes, oligonucleotides of an individual array spot may capture analytes released from its most proximal overlaying cell, as well as from cells that are not most proximal and do not overlay the spot.

Resolution of spatial cell analysis systems is dependent upon analytes being released from cells and captured by barcoded oligonucleotides on the substrate underneath or as proximate to the cells from which they originate as possible. In some examples of good resolution, captured analytes are associated with cells of the overlying tissue. When analytes diffuse to non-proximal spots and/or when oligonucleotides of individual spots capture analytes from more than just the cell contacting or overlying it, resolution can be decreased. Under these circumstances, captured analytes may not be confidently associated with individual cells of the overlying tissue.

In addition to migration of analytes to array spots and oligonucleotides of the spots, there are multiple computational components of spatial transcriptomics systems that are relevant, some examples of which are described below. In some examples, errors or inaccuracies that result from at least some of these computational components can also affect the data obtained from the spatial array and contribute to the increase or decrease in resolution of these systems.

In a first example, analytes captured by oligonucleotides on an array are associated with or assigned to a location on the array. Generally, a captured analyte is associated with a particular array spot on an array via the spatial barcode of the oligonucleotide to which it hybridizes. Two oligonucleotides that are part of different array spots can have different spatial barcodes. These spatial barcodes are generally incorporated into cDNAs made from captured mRNA templates (see FIG. 4), become part of the library made from the array, and are decoded when the nucleotide sequence of the library is obtained. For example, analytes are captured by the barcoded oligonucleotides and DNA libraries are made from cDNA copies of the analytes. The DNA libraries can be sequenced and from that sequence information the identity of the mRNA, its location on the spatial array via the spatial barcode, and the number of copies (e.g., gene expression level) of the mRNA via the associated unique molecular identifier can be determined by using an analysis pipeline such as the Space Ranger software (10× Genomics) (https://support.10×genomics.com/spatial-gene-expression/software/pipelines/latest/what-is-space-ranger).

For example, the Space Ranger software analysis pipeline can take previously obtained brightfield or fluorescence microscope images of the biological sample and overlay the gene expression data from the capture mRNA for visualization in the Loupe Browser (10× Genomics). The resolution of the spatial gene expression visualization is tied to the ability of the analytes to migrate to the proximal barcoded oligonucleotide capture probes. As such, if the analytes diffuse away from the proximal capture probes, resolution can be compromised.

As such, it would be advantageous to ensure that as many analytes as possible that are released from cells are captured by the spatial analysis systems. It would also be advantageous if analytes were captured by probes located near to the cells that release the analytes (i.e., to decrease analyte diffusion, migration distance).

### Oligonucleotide Capture Probe Assemblies

Disclosed herein are assemblies of oligonucleotides that provide extended and/or branched oligonucleotide capture probes for spatial array systems commonly used for spatial transcriptomics analyses. Assembly of the oligonucleotides generally relies on a first oligonucleotide probe having a nucleotide sequence to which a complementary nucleotide sequence in a second oligonucleotide probe can hybridize. The second oligonucleotide probe may also have an additional nucleotide sequence to which a complementary nucleotide sequence in a third oligonucleotide probe can hybridize. The third oligonucleotide probe may have an additional sequence to which a fourth oligonucleotide probe can hybridize, and so on. Hybridization of the first, second, third, fourth oligonucleotides, and so on, extends the length of oligonucleotide capture probes, as are found attached to supports (e.g., slides, beads) in conventional transcriptomics systems. Hybridization of the probes may also create branching of the assemblies, as described below.

In some examples, an oligonucleotide capture probe assembly may include a spatial array with oligonucleotide capture probes attached to a support, and successive “layers” of second, third and fourth oligonucleotides hybridized thereon. Here, the oligonucleotide capture probes attached to the support may be referred to as the first or base “layer” of oligonucleotides. A population of second oligonucleotides hybridized to the first layer may be referred to as the “second layer” of oligonucleotides, and so on. In some examples, the last “layer” of oligonucleotides may be referred to as the “final” layer. Layers of oligonucleotides added to but not including the base layer, and not including the oligonucleotides of the final layer, may be referred to as “intermediate” layers of oligonucleotides. For example, in an assembly where there is a base layer and 3 additional layers of oligonucleotides added to the base layer, the oligonucleotides of the second and third layers may be called oligonucleotides of the intermediate layers. In this example, the oligonucleotides of the fourth layer may be referred to as oligonucleotides of the final layer of oligonucleotides. Generally, oligonucleotides of the base layer have at least one hybridizing nucleotide sequence complementary to a hybridizing nucleotide sequence in the oligonucleotides of the second layer of oligonucleotides. The oligonucleotides of the second layer may have at least a second hybridizing nucleotide sequence complementary to a hybridizing nucleotide sequence in the oligonucleotides of the third layer, and so on. Oligonucleotides of the final layer of oligonucleotides may have only one hybridizing nucleotide sequence that is complementary to a hybridizing nucleotide sequence in the oligonucleotides of the last layer of oligonucleotides in the intermediate layers of oligonucleotides, but additional hybridizing nucleotide sequences may be present. In some examples, one or more oligonucleotides of the base, intermediate and/or final layers of oligonucleotides may also have a capture domain sequence for capturing analytes.

In some examples, the duplexes formed when a population of oligonucleotides making up a layer in an assembly hybridizes with a population of oligonucleotides making up a successive layer have similar melting temperatures or Tₘs. In some examples, as successive layers of oligonucleotides form duplexes with prior layers to form the oligonucleotide assemblies, the Tₘs of duplexes for successive layers decrease the farther is the layer from the base layer. For example, the Tₘ for the duplexes formed when the base layer of oligonucleotides hybridizes with a second layer of oligonucleotides may be higher than is the Tₘ for the duplexes formed between the second layer of oligonucleotides and the third layer of oligonucleotides, and so on.

The progressive decrease in Tₘ for duplexes formed between layers of oligonucleotides, the farther are the duplexed layers from the base layer, is a feature that can be advantageously used when making multi-layered assemblies of oligonucleotides. In this process, an oligonucleotide assembly can be built by simultaneously adding populations of oligonucleotides that will make up multiple layers of the assembly under conditions in which hybridization can occur. By starting the assembly process at a relatively higher temperature (at a temperature around the Tₘ for duplexes formed with the base layer) and then gradually decreasing the temperature over time, oligonucleotides can hybridize layer by layer, according to their Tₘs (i.e., oligonucleotides don't typically form duplexes at temperatures above their Tₘ) and the successive oligonucleotide layers can be built. Alternatively, the assembly process may be performed in a stepwise manner, by adding and hybridizing oligonucleotides of one layer at a time, before the successive layer is added and hybridized.

In the oligonucleotide assemblies, there may be different arrangements of features possessed by the oligonucleotides that make up the assemblies. In some examples, oligonucleotides of the base, intermediate and final layers of an assembly may all have spatial barcodes. In an example where a location along the surface of an oligonucleotide array is described in terms of x and y coordinates, and successive layers of an oligonucleotide assembly built on the array surface are described in terms of z coordinates, oligonucleotides of an assembly may each have one or more spatial barcodes designating its x-y coordinates, and a separate spatial barcode designating its z coordinate within the assembly. In some examples, oligonucleotides of an assembly may have one or the other of a spatial barcode designating x-y coordinates and z coordinates. In some examples, oligonucleotides of one or more of the base, intermediate and final layers of an assembly may not contain spatial barcodes. Other arrangements of spatial barcodes within the oligonucleotide assemblies are contemplated.

In some examples, oligonucleotides of the base, intermediate and final layers of an assembly may all have capture domains and unique molecular identifiers (UMIs). In some examples, oligonucleotides of one or more of the base, intermediate and final layers of an assembly may not have capture domains and UMIs. In some examples, oligonucleotides of only the final layer of an assembly may have capture domains and UMIs. Other arrangements of capture domains and UMIs within the oligonucleotide assemblies are contemplated.

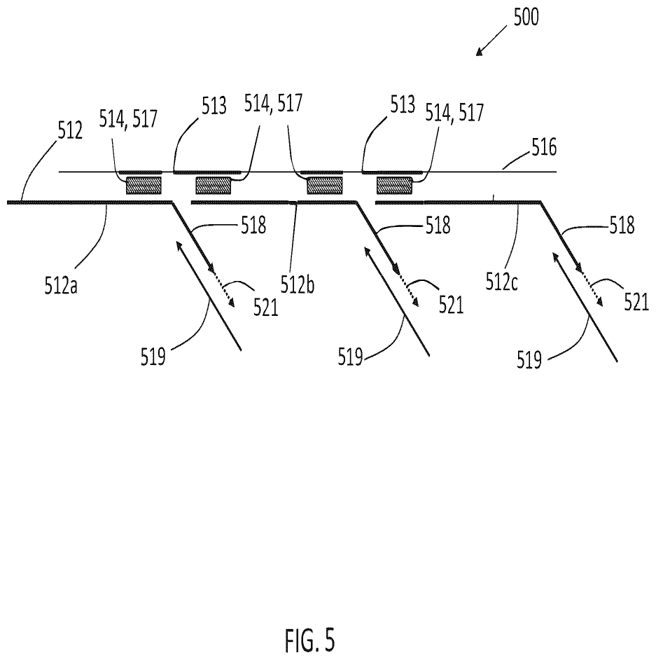

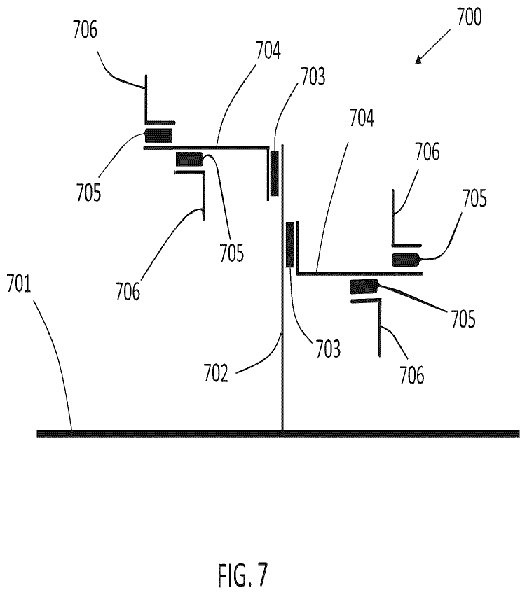

In this system, when oligonucleotides that comprise a layer have a single iteration of a nucleotide sequence capable of hybridizing to oligonucleotides of a successive oligonucleotide layer, an extended oligonucleotide assembly may be built out or “extended” without branching (e.g., as in FIGS. 5, 6 and 8). In contrast, when oligonucleotides that comprise a layer have two or more iterations of a sequence or sequences to which oligonucleotides of a second layer may hybridize, the successive layer of oligonucleotides may be “branched” (e.g., as in FIG. 7).

For example, when oligonucleotides of a first layer each have two iterations of nucleotide sequences to which oligonucleotides of a second layer may hybridize, two of the second-layer oligonucleotides will hybridize to each oligonucleotide of the first layer. Conceptually, then, the total number of oligonucleotides in the second layer is twice the number of oligonucleotides in the first layer. If each oligonucleotide of the second layer also has two iterations of a sequence to which an oligonucleotide of the third layer may hybridize, the oligonucleotides in the third layer may again double. In this scenario, there is a branching or “amplification” of capture probes in the consecutive layers. In this example, the amplification is two-fold in each successive layer (e.g., from 1 to 2 to 4). Increasing the number of nucleotide sequences to which oligonucleotides in a successive layer may bind, from 2 as shown here, to 3, 4, 5, 6 or more, can result in higher levels of branching or amplification in such oligonucleotide assemblies. Increased branching of the oligonucleotide layers can increase the density of capture domains in an array.

Various nonlimiting embodiments of the oligonucleotide assemblies and the oligonucleotides that comprise the assemblies are described below.

FIG. 5 schematically illustrates a first exemplary embodiment of a spatially barcoded oligonucleotide capture probe assembly 500 that includes a branched mRNA-capturing probe 512 extending from a slide, bead or other carrier (not shown) and having a plurality of barcoded hybridization regions 514 arranged in a straight chain 516 (the box labeled 514 indicates hybridization between the single strands located on either side of the box). As illustrated, the branched mRNA-capturing probe 512 includes a plurality of segments 512a, 512b, 512c, wherein each segment includes at least a portion of an oligonucleotide sequence. Each hybridization region 514 includes at least a portion of an oligonucleotide sequence contained in segment 512a, 512b or 512c, which can be the sequence complementary to a portion of a bridging element in the embodiment shown. A bridging element 513 hybridizes to and joins the adjacent segments together and maintains the directional orientation of the segments. The bridging element 513 can include at least a portion of an oligonucleotide sequence, a polymer, a crosslinking chemical or synthetic molecule that preserves a 5′ to 3′ orientation of the oligonucleotide segments 512a, 512b and 512c.

A barcoded region 517, such as a spatially barcoded region, at least partially overlaps the hybridization regions 514 and can be entirely within the hybridization regions 514. An mRNA-capturing branch 518 extends from each of a plurality of hybridization regions 514 and at least part of its sequence is used to capture mRNA 519, which can be used as a template to extend the mRNA-capturing branch 518 with a polymerase to make cDNA 521. The mRNA-capturing branches 518 can include additional oligonucleotide sequences and can be joined to the oligonucleotide segments 512a, 512b and 512c by attaching the 3′ ends of the mRNA-capturing branches 518 to the 5′ end of the oligonucleotide segments 512a, 512b and/or 512c (as shown), or by attaching the 5′ end of the mRNA-capturing branch 518 to the 3′end of the oligonucleotide segment 512a, 512b and/or 512c. In the drawing, the arrowheads represent the 3′ends of the oligonucleotide segments 512a, 512b, 512c and the mRNA-capturing branches 518, and the respective 5′ends are opposite the respective arrowheads. As used herein, the term “plurality” means “two or more” and the term each” means “each of the two or more.” For example, if a hybridization region contains ten nucleotides and six of the ten nucleotides have mRNA-capturing branches extending from them, then those six nucleotides would constitute a “plurality” for purposes of this invention.

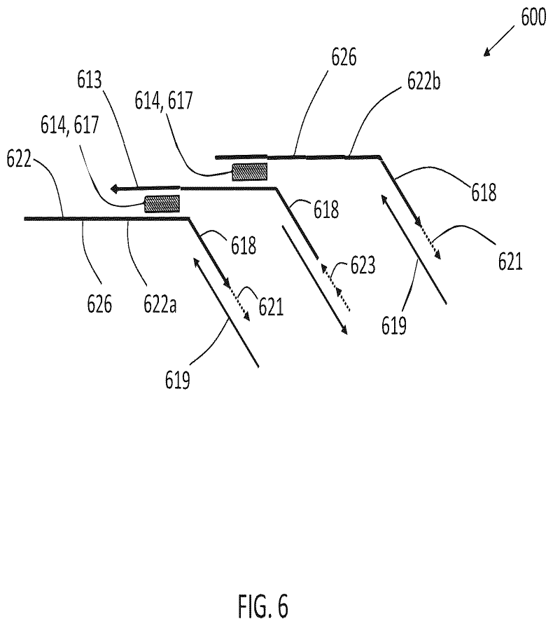

FIG. 6 schematically illustrates a second exemplary embodiment of an oligonucleotide capture probe assembly 600 of the invention. The oligonucleotide capture probe assembly 600 includes a branched mRNA-capturing probe 622 extending from a slide, bead or other carrier (not shown) and having a plurality of barcoded hybridization regions 614 arranged in a stepped (cascaded) chain 626. As illustrated, the branched mRNA-capturing probe 622 includes a plurality of stepped segments 622a, 622b, wherein each segment includes at least a portion of an oligonucleotide sequence. Each hybridization region 614 includes at least a portion of an oligonucleotide sequence contained in segment 622a or 622b, which can be sequences complementary to a bridging element in the embodiment shown. A bridging element 613 hybridizes to and joins the adjacent segments 622a and 622b together and maintains them in a stepped configuration as well as in a directional orientation of the segments. The bridging element 613 can include at least a portion of an oligonucleotide sequence, a polymer, a crosslinking chemical or synthetic molecule that preserves a 5′-3′ orientation of the oligonucleotide segments 622a and 622b.

A barcoded region 617, such as a spatially barcoded region, at least partially overlaps the hybridization regions 614 and can be entirely within the hybridization regions 614. An mRNA-capturing branch 618 extends from each of a plurality of hybridization regions 614 and at least part of its sequence is used to capture mRNA 619, which can be used as a template to extend the mRNA-capturing branch 618 with a polymerase to make cDNA 621. The mRNA-capturing branches 618 can include additional oligonucleotide sequences and can be joined to the oligonucleotide segments 622a and 622b by attaching the 3′ ends of the mRNA-capturing branches 618 to the 5′ end of the oligonucleotide segments 622a and 622b (as shown), or by attaching the 5′ end of the mRNA-capturing branch 618 to the 3′end of the oligonucleotide segment 622a and 622b. In the drawing, the arrowheads represent the 3′ ends of the oligonucleotide segments 622a and 622b and the mRNA-capturing branches 618, and the respective 5′ends are opposite the respective arrowheads. The splint oligonucleotide in the bridging element 613 can include a 5′ capture sequence 618′ in the capturing branch 618, which can be used to capture targeted mRNA's, and which can be extended in the 5′ direction through ligation of degenerated oligonucleotides, for example.

During use, the spatially barcoded oligonucleotide capture probe assembly 500, 600 is applied to a microscopic slide, such as a spatial gene expression slide. Alternatively, a base layer of oligonucleotides is attached to the slide and the successive layers are built through hybridization. Building of the oligonucleotide assemblies occurs before a tissue section has been applied to the surface of the spatial array.

Sections of biological tissue are arranged on the slide. The sections of tissue have mRNA molecules both at the tissue surfaces and the tissue interior. When the tissue sections are permeabilized, the barcoded hybridization regions 514, 614 of the branched mRNA-capturing probe 512, 612 or 522, 622 contact the adjacent surface of each tissue section and capture mRNA molecules from that tissue surface. The mRNA-capturing branches 518, 618 which can also be barcoded, extend into the interior of each tissue section and capture mRNA molecules from the interior. When the tissue sections are removed from the slide and the slide is imaged, the image will reflect mRNA molecules both at the tissue surface and the tissue interior. Because the tissue interior constitutes most of the tissue section, the resulting image can more accurately reflect the types of mRNA that exist in the tissue section and may not exist at the surface. This can result in better detection of diseases that are represented by RNA defects, including without limitation heart disease, some cancers, stroke, myotonic dystrophy, some neurogenerative diseases, and others.

### Physical Characteristics of the Oligonucleotide Capture Probes

The oligonucleotide capture probes can be characterized both in terms of physical characteristics and chemistry. Physical characteristics are important in order to ensure adequate penetration of the mRNA-capturing branches into the interior of the permeabilized biological tissue. The length of a biological molecular chain can be characterized in terms of nucleotides or base units for a single stranded RNA or double stranded DNA (with one nucleotide=one base unit), or base pairs for a double stranded DNA, and the length of a mRNA-capturing branches can be similarly characterized.

The length of a base pair for a double-stranded DNA molecule is about 3.4 angstroms. Permeabilized tissue sections typically have a thickness of about 2 microns to about 20 microns, more commonly about 5 microns to about 15 microns. Preferably, tissue sections are approximately 10 microns thick. For a DNA-based mRNA capture branch to completely pass through a typical biological tissue section would therefore require a branch length of about 5880 base pairs for a 2-micron tissue section, about 14,700 base pairs for a 5-micron tissue section, about 44, 100 base pairs for a 15-micron tissue section, and about 58,800 base pairs for a 20-micron tissue section. The lengths of single-stranded RNA vary more widely, often between about 1500 and about 4500 nucleotides per micron depending on the type of RNA. Such long branches present difficulties in synthesis and can result in steric hindrance. Therefore, optimal performance of the oligonucleotide capture probe typically results using mRNA-capturing branches that probe into the tissue sections but do not necessarily pass through all of the thickness of the tissue sections.

Due to limitations in synthesis and availability, the oligonucleotide capture probe may have an overall chain length of about 20 to about 20,000 nucleotides, or about 100 to about 15,000 nucleotides, or about 1000 to about 8000 nucleotides. Thus, for a mRNA-capturing branch to penetrate completely through the permeabilized biological tissue, the length of the mRNA-capturing branches would have to be large relative to the chain length of the oligonucleotide capture probe. This can be impractical because excessively long branches might fold or curl on or near the tissue surface instead of accessing the interior of the tissue. Moreover, excessively long branches might entangle and/or hinder each other and obstruct contact between the tissue surface and the barcoded hybridization regions of the oligonucleotide capture probe.

For these reasons, the mRNA-capturing branches should be designed to penetrate as far as possible into the biological tissue while avoiding the problems associated with excessive length. The mRNA-capturing branches may have lengths of at least about 5 nucleotides, or at least about 10 nucleotides, or at least about 20 nucleotides, or at least about 50 nucleotides, or at least about 100 nucleotides, or at least about 250 nucleotides, or at least about 500 nucleotides, or at least about 750 nucleotides, or at least about 1000 nucleotides. The optimal length may vary depending on the overall chemistry of the oligonucleotide capture probe, the relative rigidity of the mRNA-capturing branches, the type and thickness of the permeabilized biological tissue, and other factors.

The oligonucleotide capture probe includes a plurality of hybridized regions arranged in a chain. The hybridized regions are suitably barcoded and are suitably spatially barcoded. In one embodiment, it may be desirable to include as many as possible hybridized regions per the overall chain length of the oligonucleotide capture probe. This embodiment would enable a high number of mRNA-capturing branches extending from the hybridized regions, especially where an mRNA-capturing branch extends from each of the hybridized regions. In this embodiment, the hybridized regions of the oligonucleotide capture probe may have individual lengths of about 5 to about 2000 nucleotides, or about 10 to about 1000 nucleotides, or about 20 to about 500 nucleotides, or about 25 to about 100 nucleotides. In another embodiment, it may be desirable for the hybridized regions to have lengths like or exceeding the lengths of the mRNA-capturing branches, so that the mRNA-capturing branches do not significantly hinder contact between the hybridized regions and the surfaces of the permeabilized tissue sections. In this embodiment, the hybridized regions may have lengths of at least about ten nucleotides, or at least about 20 nucleotides, or at least about 50 nucleotides, or at least about 100 nucleotides, or at least about 250 nucleotides, or at least about 500 nucleotides, or at least about 750 nucleotides, or at least about 1000 nucleotides.

### Chemistry of the Oligonucleotide Capture Probe

Referring to FIGS. 5 and 6, the exemplary spatially barcoded oligonucleotide capture probe 500 or 600 includes a branched mRNA-capturing probe 512 or 622 having a plurality of barcoded hybridization regions 514 arranged in a straight chain 516 or stepped chain 626. A plurality of mRNA-capturing branches 518, 618 extend from the hybridization regions 514, 614. The hybridization regions 514, 614 arranged in the chain 516 or 626 serve as capture domains for target mRNA present on the adjacent surface of the tissue sections. Each capture domain can be an oligonucleotide, a polypeptide, a small molecule, or any combination thereof, that binds, captures and/or detects a target mRNA.

### Chemistry of Capture Regions

Each capture domain can be a functional nucleic acid sequence configured to interact with the target mRNA molecules. The functional sequence can include a poly(T) sequence, which poly(T) sequences are configured to interact with the mRNA molecules via the poly(A) tail of an mRNA transcript.

Capture domains can include ribonucleotides and/or deoxyribonucleotides as well as synthetic nucleotide residues that can participate in Watson-Crick type or analogous base pair interactions. The capture domains can prime a reverse transcription reaction to generate cDNA that is complementary to the captured mRNA molecules.

A capture domain can be located at the end of the capture probe and can include a free 3′ end that can be extended, e.g., by template dependent polymerization, to form an extended capture probe. In some embodiments, the capture domain includes a nucleotide sequence that is capable of hybridizing to mRNA present in the cells of the biological tissue contacted with the array. The capture domain can be selected or designed to bind selectively or specifically to a target mRNA by way of hybridization to the mRNA poly(A) tail. Thus, the capture domain can include a poly(T) DNA oligonucleotide, e.g., a series of consecutive deoxythymidine residues linked by phosphodiester bonds, which is capable of hybridizing to the poly(A) tail of mRNA. The capture domain can include nucleotides that are functionally or structurally analogous to a poly(T) tail, for example, a poly(U) oligonucleotide or an oligonucleotide including deoxythymidine analogues. The capture domain can have a sequence that is capable of binding to mRNA. For example, the capture domain can include a nucleic acid sequence (e.g., a poly(T) sequence) capable of binding to a poly(A) tail of an mRNA. In some embodiments, a homopolymer sequence is added to an mRNA molecule using a terminal transferase enzyme in order to produce a molecule having a poly(A) or poly(T) sequence. For example, a poly(A) sequence can be added to an mRNA, thereby making the mRNA capable of capture by a poly(T) capture domain.

In some embodiments, random sequences, e.g., random hexamers or similar sequences, can be used to form all or a part of the capture domain. Example, random sequences can be used in conjunction with poly(T) (or poly(T) analogue) sequences. Thus, where a capture domain includes a poly(T) (or a “poly(T)-like”) oligonucleotide, it can also include a random oligonucleotide sequence (e.g., “poly(T)-random sequence” probe). This can, for example, be located at 5′ or 3′ of the poly(T) sequence, e.g., at the 3′ end of the capture domain. The poly(T)-random sequence probe can facilitate the capture of the mRNA poly(A) tail. In some embodiments, the capture domain can be an entirely random sequence. In some embodiments, degenerate capture domains can be used.

In some embodiments, a pool of two or more capture probes form a mixture, where the capture domain of one or more capture probes includes a poly(T) sequence and the capture domain of one or more capture probes includes random, semi-random or non-random sequences. In some embodiments, a pool of two or more capture probes form a mixture where the capture domain of one or more capture probes includes a poly(T)-like sequence and the capture domain of one or more capture probes includes random, semi-random or non-random sequences. In some embodiments, probes with degenerate capture domains can be added to any of the preceding combinations listed herein. In some embodiments, probes with degenerate capture domains can be substituted for one of the probes in each of the pairs described herein.

The capture domain can be based on a gene sequence, a motif sequence or common/conserved sequence that it is designed to capture (i.e., a sequence-specific capture domain). Thus, the capture domain can be capable of binding selectively to a desired sub-type or subset of nucleic acid, for example a type or subset of mRNA. In some embodiments, a capture domain includes an “anchor” or “anchoring sequence,” which is a sequence of nucleotides designed to ensure that the capture domain hybridizes to the intended mRNA. The anchor sequence can include a sequence of nucleotides, including a 1-mer, 2-mer, 3-mer or longer sequence. The sequence can be random. For example, a capture domain including a poly(T) sequence can be designed to capture an mRNA. An anchoring sequence can include a random 3-mer (e.g., GGG) that helps ensure that the poly(T) capture domain hybridizes to an mRNA. In some embodiments, an anchoring sequence can be VN, N, or NN. Alternatively, the sequence can be designed using a specific sequence of nucleotides. In some embodiments, the anchor sequence is at the 3′ end of the capture domain. In some embodiments, the anchor sequence is at the 5′ end of the capture domain.

Referring to FIG. 5, the mRNA-capturing branches 518 extend from the hybridization regions 514, can be barcoded, and can serve the same chemical purpose of the capture domains that define the hybridization regions. The primary difference is that the hybridization regions 514 capture mRNA from the adjacent surface of the tissue section, whereas the mRNA-capturing branches 518 capture mRNA that is present in the interior of the tissue section.

In the exemplary embodiments shown in FIGS. 5 and 6, the hybridization regions 514, 614 and the mRNA-capturing branches 518, 618 can each be formed of the same barcoded mRNA-capturing oligonucleotide, which can be a bar-coded oligo-dT oligonucleotide.

### Barcode Chemistry

Referring to FIGS. 5 and 6, each hybridized region 514, 614 and each mRNA-capturing branch 518, 618 may include a barcode 517, 617, which can suitably be a spatial barcode. The mRNA-capturing probe 512 or 622 can thus include several spatial barcodes, for example, barcodes corresponding to each of the hybridized regions 514, 614 and each of the mRNA-capturing branches 518, 618. A “spatial barcode” is a contiguous nucleic acid segment or two or more non-contiguous nucleic acid segments that function as a label or identifier that conveys or can convey spatial information. In some embodiments, the spatial barcodes are associated with locations within the array or tissue section.

A spatial barcode can function both as a spatial barcode and as a unique molecular identifier (UMI), associated with a capture probe or capture domain. Spatial barcodes can have a variety of different formats. For example, spatial barcodes can include polynucleotide spatial barcodes, random nucleic acid and/or amino acid sequences, and synthetic nucleic acid and/or amino acid sequences. In some embodiments, a spatial barcode is attached to an mRNA before, during, and/or after sequencing. The spatial barcode can allow for identification and/or quantification of individual sequencing-reads. The spatial barcode can be a fluorescent barcode for which fluorescently labeled oligonucleotide probes hybridize to the spatial barcode. The spatial barcode can be a nucleic acid sequence that does not substantially hybridize to mRNA molecules in a biological tissue. In some embodiments, the spatial barcode has less than 80% sequence identity (e.g., less than 70%, 60%, 50%, or less than 40% sequence identity) to the nucleic acid sequences across a substantial part (e.g., 80% or more) of the mRNA molecules in the biological tissue. The spatial barcode sequences can include from about 5 to about 20 or more nucleotides within the sequence of the mRNA-capturing probe. For example, the length of a spatial barcode sequence can be about 6, 7, 8, 9, 10, 11, 12, 13, 14, 15, 16, 17, 18, 19, 20 nucleotides or longer. In some embodiments, the length of a spatial barcode sequence can be at least about 6, 7, 8, 9, 10, 11, 12, 13, 14, 15, 16, 17, 18, 19, 20 nucleotides or longer, or at most about 6, 7, 8, 9, 10, 11, 12, 13, 14, 15, 16, 17, 18, 19, 20 nucleotides or shorter. However, while a spatial barcode could also function as a UMI, a UMI could also be a separate sequence found on the capture probe. As such, a capture probe could have both a spatial barcode and a UMI, which are of different sequence.

These nucleotides can be completely contiguous, e.g., in a single stretch of adjacent nucleotides, or they can be separated into two or more subsequences that are separated by 1 or more nucleotides. Separated spatial barcode subsequences can be from about 4 to about 16 nucleotides in length. In some embodiments, the spatial barcode subsequence can be about 4, 5, 6, 7, 8, 9, 10, 11, 12, 13, 14, 15, 16 nucleotides or longer, or at least about 4, 5, 6, 7, 8, 9, 10, 11, 12, 13, 14, 15, 16 nucleotides or longer, at most about 4, 5, 6, 7, 8, 9, 10, 11, 12, 13, 14, 15, 16 nucleotides or shorter. For multiple capture probes that are attached to a common array feature, the one or more spatial barcode sequences of the multiple capture probes can include sequences that are the same for all capture probes coupled to the feature, and/or sequences that are different across all capture probes coupled to the feature.

### Synthesis of the Oligonucleotide Capture Probe Assembly

Referring to FIGS. 5 and 6, a key to the spatially barcoded oligonucleotide capture probe assembly 500 or 600 is the branched mRNA-capturing probe 512 or 622 having a plurality of hybridization regions 514 arranged in a straight 516 or stepped chain 626, and an mRNA-capturing branch 518, 618 extending from each hybridization region 514, 614. The dashed lines 521, 621 in the mRNA-capturing branches 518, 618 represent a typical reverse transcription product, namely cDNA. The dashed lines 623 with arrows (FIG. 6) reflect the use of chemical ligation to attach and/or lengthen the mRNA-capturing branches. The mRNA-capturing branches can be attached to the barcoded hybridization regions using chemical ligation and can also be barcoded. Chemical ligation connects two oligonucleotides, in this case a capture domain and an mRNA-capturing branch, by covalent bonding using a third oligonucleotide called a template strand, typically in the presence of a condensing reagent. The two oligonucleotides are assembled on the template in opposite directions by forming antiparallel and parallel duplexes simultaneously, followed by coupling with a condensing reagent such as N-cyanoimidazole under mild conditions. The ligation reaction results in the formation of 3′ to 3′ or 5′ to 5′ ester bonds between the two oligonucleotides.

Specific examples of chemical ligation are described in detail in U.S. Pat. No. 7,264,929, the disclosure of which is incorporated by reference. Chemical ligation has commonly been used to form longer chain oligonucleotides including, for example, longer chain cDNA molecules. The present invention applies the chemical ligation techniques to synthesize branched mRNA-capturing probes useful in spatial transcriptomics, by attaching the branched mRNA-capturing domains to the capture domains of straight-chain oligonucleotides such as those used in conventional reverse transcription primers. The capture domains in the main chain and the mRNA-capturing branches can be barcoded using conventional techniques. The barcoding can be accomplished before or after the chemical ligation of the mRNA-capturing branches to the capture domains in the primary chain.

### Branched Oligonucleotide Assemblies

In other examples, the oligonucleotide capture probe assemblies can be designed to increase the density of capture domains within an area. FIG. 7 is a schematic representation of such an embodiment. FIG. 7 illustrates an example oligonucleotide capture probe assembly 700. In the assembly 700, a first oligonucleotide 702 is attached to a support 701. The support 700 may be part of a spatial array.

The first oligonucleotide 702 contains at least two nucleotide sequences 703 that are complementary to sequences in a second oligonucleotide 704 (note that rectangles designated as 703 represent hybridization between 702 and 704). Two second oligonucleotides 704 hybridize to the single first oligonucleotide 702 via the nucleotide sequences 703. Each of the second oligonucleotides 704 contain two nucleotide sequences 705 that are complementary to sequences in a third oligonucleotide 706 (note that the rectangles designated as 705 represent hybridization between 704 and 706). Two third oligonucleotides 706 hybridize to each second oligonucleotide 704 via the nucleotide sequences 705.

This embodiment demonstrates how increasing, in an oligonucleotide, the number of nucleotide sequences to which a second oligonucleotide can hybridize, “amplifies” the number of oligonucleotides and, in some examples, the number of capture domains in the oligonucleotides, within an area or volume. In the example here, two second oligonucleotides hybridize to a single first oligonucleotide. In the example, two third oligonucleotides hybridize to each of the two illustrated second oligonucleotides. Therefore, a single first oligonucleotide is “amplified” to two second oligonucleotides, which are amplified to four third oligonucleotides (1+2+4). One can alter this amplification and the density of oligonucleotides within an assembly by changing the number of hybridizing nucleotide sequences within oligonucleotides of the system. The invention contemplates 1, 2, 3, 4, 5, 6 or more hybridizing nucleotide sequences within oligonucleotides used to form the oligonucleotide assemblies described herein. By increasing the number of oligonucleotides in the assembly, the number of capture domains can also be increased. For example, oligonucleotide 706 can each comprise a capture domain in the unhybridized region. In viewing FIG. 7, one can see from one oligonucleotide affixed to the array substrate (702) there are found 4 potential capture domains, or free ends of the oligonucleotide 706 that are available to capture target analytes.

### Duplex Melting Temperatures in Oligonucleotide Assemblies

In another feature of the disclosed oligonucleotide assemblies, the nucleotide sequences within oligonucleotides of the assemblies that hybridize to other oligonucleotides (i.e., hybridizing nucleotide sequences), thus building the successive “layers” of the assemblies, may be designed to have different melting temperatures or Tₘ's. In some examples, the Tₘ's for hybridization of the oligonucleotides of one layer to oligonucleotides of a successive layer may be the same. This feature provides for formation of duplexes between all the oligonucleotides of the two intersecting layers at the same temperature and at the same time when the oligonucleotide assemblies are built.

In addition to the feature of duplexes between layers of oligonucleotides having about the same Tₘ, the oligonucleotides of an assembly may be designed such that Tₘ's of duplexes between two layers decrease the farther from the base layer the duplexes are located. For example, the Tₘ for duplexes formed between the second and third layers of oligonucleotides in an assembly may be higher than the Tₘ for duplexes formed between the third and fourth layer of oligonucleotides in the assembly. Similarly, the Tₘ for duplexes formed between the third and fourth layers of oligonucleotides in an assembly may be higher than the Tₘ for duplexes formed between the fourth and fifth layers of oligonucleotides in the assembly, and so on.

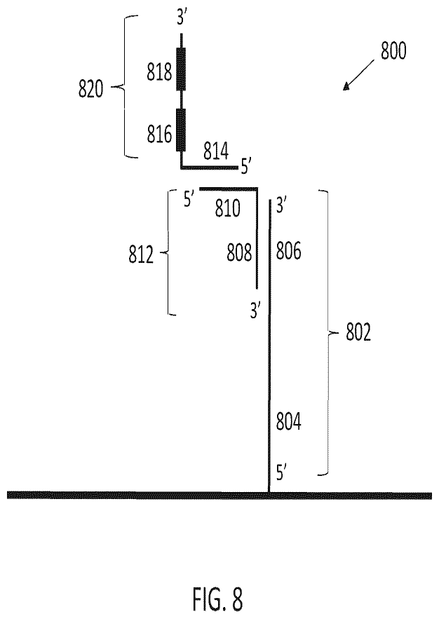

FIG. 8 illustrates an example of this. First, an array with a plurality of first probes and a biological sample disposed on the array is provided. The plurality of first probes is affixed to the array surface and a first probe 802 of the plurality of first probes includes a first hybridization sequence 806. Second, the array is contacted with a biological sample, a plurality of second probes, and a plurality of third probes. The second probe 812 of the plurality of second probes comprises in a 3′ to a 5′ direction: (i) a second hybridization sequence 808 that specifically binds to the first hybridization sequence 806 in the first probe at a temperature that is about or less than a first melting temperature, and (ii) a third hybridization sequence 810. A third probe 820 of the plurality of third probes comprises in a 5′ to a 3′ direction: (i) a fourth hybridization sequence 814 that specifically binds to the third hybridization sequence 810 in the second probe at a temperature that is about or less than a second melting temperature, where the first melting temperature is higher than the second melting temperature, (ii) a spatial barcode 816, and (iii) a capture domain 818 that specifically binds to a target analyte. A first probe of the plurality of first probes may comprise a first probe spatial barcode 804.

In some embodiments, by placing the biological sample with array and the first, second, and third probes, and decreasing the temperature from a temperature of about or less than the first melting temperature to a temperature of about or less than the second melting temperature, the first, second, and third probes assemble to create an organized structure that ‘invades’ the biological sample in an organized fashion in a direction perpendicular from the two-dimensional surface of the array (z-direction), while remaining affixed to the array. In other embodiments, the structures are built on the array, following a pattern of decreasing temperature as described above, prior to placing the biological sample on the array.

### Additional Embodiments

Regarding the features of the oligonucleotide assemblies described herein, the methods and systems may include the use of an array (e.g., any of the exemplary arrays described herein), where the array has a plurality of first probes affixed (i.e., attached) to the array (i.e. reversibly or non-reversibly).

A first probe of the plurality of first probes can include a first hybridization sequence.

The first hybridization sequence can have a total of about 5 nucleotides to about 100 nucleotides, about 5 nucleotides to about 90 nucleotides, about 5 nucleotides to about 80 nucleotides, about 5 nucleotides to about 70 nucleotides, about 5 nucleotides to about 60 nucleotides, about 5 nucleotides to about 50 nucleotides, about 5 nucleotides to about 40 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 100 nucleotides, about 10 nucleotides to about 90 nucleotides, about 10 nucleotides to about 80 nucleotides, about 10 nucleotides to about 70 nucleotides, about 10 nucleotides to about 60 nucleotides, about 10 nucleotides to about 50 nucleotides, about 10 nucleotides to about 40 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 100 nucleotides, about 15 nucleotides to about 90 nucleotides, about 15 nucleotides to about 80 nucleotides, about 15 nucleotides to about 70 nucleotides, about 15 nucleotides to about 60 nucleotides, about 15 nucleotides to about 50 nucleotides, about 15 nucleotides to about 40 nucleotides, about 15 nucleotides to about 30 nucleotides, about 15 nucleotides to about 25 nucleotides, about 15 nucleotides to about 20 nucleotides, about 20 nucleotides to about 100 nucleotides, about 20 nucleotides to about 90 nucleotides, about 20 nucleotides to about 80 nucleotides, about 20 nucleotides to about 70 nucleotides, about 20 nucleotides to about 60 nucleotides, about 20 nucleotides to about 50 nucleotides, about 20 nucleotides to about 40 nucleotides, about 20 nucleotides to about 30 nucleotides, about 20 nucleotides to about 25 nucleotides, about 25 nucleotides to about 100 nucleotides, about 25 nucleotides to about 90 nucleotides, about 25 nucleotides to about 80 nucleotides, about 25 nucleotides to about 70 nucleotides, about 25 nucleotides to about 60 nucleotides, about 25 nucleotides to about 50 nucleotides, about 25 nucleotides to about 40 nucleotides, about 25 nucleotides to about 30 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60 nucleotides, about 30 nucleotides to about 50 nucleotides, about 30 nucleotides to about 40 nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50 nucleotides, about 50 nucleotides to about 100 nucleotides, about 50 nucleotides to about 90 nucleotides, about 50 nucleotides to about 80 nucleotides, about 50 nucleotides to about 70 nucleotides, about 50 nucleotides to about 60 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 100 nucleotides, about 70 nucleotides to about 90 nucleotides, about 70 nucleotides to about 80 nucleotides, about 80 nucleotides to about 100 nucleotides, about 80 nucleotides to about 90 nucleotides, or about 90 nucleotides to about 100 nucleotides. Second, third, fourth, fifth hybridization sequences, and so on, may have similar properties.

In some embodiments of any of the methods described herein, the first probe can also include a spatial barcode positioned adjacent (e.g., positioned 5′) to the first hybridization sequence.

The first probe may also include a capture domain. In some examples, the capture domain can include a poly(T) sequence.

In some embodiments of any of the methods described herein, the first probe can also include a cleavage domain (e.g., any of the exemplary cleavage domains described herein) positioned adjacent (e.g., positioned 5′) to the spatial barcode, if the spatial barcode is present on the first probe. In some embodiments, the cleavage domain can be a disulfide bond that is cleaved by inducing reducing conditions.

The array to which the first probes are affixed may have any of the characteristics of any of the arrays described herein. For example, the plurality of first probes can be printed on, or attached to, the array in designated spots, wherein the spots are separated from each other by a distance of about 20 pm, about 30 pm, about 40 pm, about 50 pm, about 60 pm, about 70 pm, about 80 pm, about 90 pm, about 100 pm, about 125 pm, about 150 pm, about 175 pm, about 200 pm, from the center of one spot to the center of the next spot.

In some embodiments of any of the methods described herein, the array includes a slide. In some embodiments, the array can be a bead array (e.g., any of the exemplary bead arrays described herein).

A second probe of the plurality of second probes comprises (e.g., in a 3′ to a 5′ direction): (i) a second hybridization sequence that specifically binds to the first hybridization sequence at a temperature that is about or less than a first melting temperature, and (ii) a third hybridization sequence. The second probe may comprise an analyte capture domain.

A third probe of the plurality of third probes comprises (e.g., in a 5′ to a 3′ direction): (i) a fourth hybridization sequence that specifically binds to the third hybridization sequence at a temperature that is about or less than a second melting temperature, where the first melting temperature is higher than the second melting temperature, (ii) a spatial barcode, and (iii) a capture domain that specifically binds to a target analyte (e.g., any of the exemplary capture domains described herein binding any of the exemplary target analytes described herein).

The first hybridization sequence and the second hybridization sequence specifically bind to each other at a temperature that is about or less than the first melting temperature. In some embodiments, the first melting temperature is about 55° C. to about 65° C., about 55° C. to about 64° C., about 55° C. to about 63° C., about 55° C. to about 62° C., about 55° C. to about 61° C., about 55° C. to about 60° C., about 55° C. to about 59° C., about 55° C. to about 58° C., about 55° C. to about 57° C., about 56° C. to about 65° C., about 56° C. to about 64° C., about 56° C. to about 63° C., about 56° C. to about 62° C., about 56° C. to about 61° C., about 56° C. to about 60° C., about 56° C. to about 59° C., about 56° C. to about 58° C., about 57° C. to about 65° C., about 57° C. to about 64° C., about 57° C. to about 63° C., about 57° C. to about 62° C., about 57° C. to about 61° C., about 57° C. to about 60° C., about 57° C. to about 59° C., about 58° C. to about 65° C., about 58° C. to about 64° C., about 58° C. to about 63° C., about 58° C. to about 62° C., about 58° C. to about 61° C., about 58° C. to about 60° C., about 59° C. to about 65° C., about 59° C. to about 64° C., about 59° C. to about 63° C., about 59° C. to about 62° C., about 59° C. to about 61° C., about 60° C. to about 65° C., about 60° C. to about 64° C., about 60° C. to about 63° C., about 60° C. to about 62° C., about 61° C. to about 65° C., about 61° C. to about 64° C., about 61° C. to about 63° C., about 62° C. to about 65° C., about 62° C. to about 64° C., or about 63° C. to about 65° C. In some embodiments, the first melting temperature can be about 58° C. to about 62° C.

The third hybridization sequence of the second probe and the fourth hybridization sequence of the third probe specifically bind to each other at a temperature that is about or less than the second melting temperature. In some embodiments, the second melting temperature is about 45° C. to about 55° C., about 45° C. to about 54° C., about 45° C. to about 53° C., about 45° C. to about 52° C., about 45° C. to about 51° C., about 45° C. to about 50° C., about 45° C. to about 49° C., about 45° C. to about 48° C., about 45° C. to about 47° C., about 46° C. to about 55° C., about 46° C. to about 54° C., about 46° C. to about 53° C., about 46° C. to about 52° C., about 46° C. to about 51° C., about 46° C. to about 50° C., about 46° C. to about 49° C., about 46° C. to about 48° C., about 47° C. to about 55° C., about 47° C. to about 54° C., about 47° C. to about 53° C., about 47° C. to about 52° C., about 47° C. to about 51° C., about 47° C. to about 50° C., about 47° C. to about 49° C., about 48° C. to about 55° C., about 48° C. to about 54° C., about 48° C. to about 53° C., about 48° C. to about 52° C., about 48° C. to about 51° C., about 48° C. to about 50° C., about 49° C. to about 55° C., about 49° C. to about 54° C., about 49° C. to about 53° C., about 49° C. to about 52° C., about 49° C. to about 51° C., about 50° C. to about 55° C., about 50° C. to about 54° C., about 50° C. to about 53° C., about 50° C. to about 52° C., about 51° C. to about 55° C., about 51° C. to about 54° C., about 51° C. to about 53° C., about 52° C. to about 55° C., about 52° C. to about 54° C., or about 53° C. to about 55° C. In some embodiments, the second melting temperature is about 48° C. to about 52° C.

In some embodiments of any of these methods, contacting of the probes to build an oligonucleotide assembly includes decreasing the temperature from a temperature of about or less than the first melting temperature to a temperature of about or less than the second melting temperature. The decreasing of the temperature from a temperature of about or less than the first melting temperature to a temperature of about or less than the second melting temperature results in the first hybridization sequence of the first probe specifically binding to the second hybridization sequence of the second probe, and the third hybridization sequence of the second probe specifically binding to the fourth hybridization sequence of the third probe. Additional probes can be hybridized using this strategy.

Additional probes and corresponding hybridization sequences can be used in a similar fashion. Additional hybridization sequences in additional probes may have similarly decreasing binding/melting temperatures.

Some embodiments of any of the methods described herein include disposing a biological sample (e.g., any biological sample described herein) onto an array (e.g., any of the exemplary arrays described herein), wherein the array has a plurality of first probes (e.g., any of the exemplary first probes described herein) affixed (i.e., attached) to the array. In some embodiments, the array is contacted with a biological sample and a plurality of second and third probes. In some embodiments, the array is contacted with the plurality of second and third probes prior to the placement of the biological sample.

In some embodiments of any of the methods described herein, the first probe, the second probe, and the third probe hybridize, assemble, and penetrate the biological sample.

In some embodiments of any of the methods described herein, the biological sample has not been treated with a permeabilization agent (e.g., any of the permeabilization agents described herein). In some embodiments, the biological sample that has not been treated with a permeabilization agent can be a fresh frozen tissue sample. In some embodiments, the biological sample has been treated with a permeabilization agent (e.g., one or more of any of the permeabilization agents described herein). In some embodiments of any of the methods described herein, the biological sample has been treated with a fixation agent (e.g., any of the fixation agents described herein).

In some embodiments of any of the methods described herein, the biological sample can be a tissue sample. In some examples of any of the methods described herein, the tissue sample can be a tissue section. For example, the biological sample can be a formalin-fixed paraffin-embedded tissue sample or a fresh frozen tissue sample. A biological sample could be a tissue sample from a plant. In some examples, a tissue sample is from a human, a non-human primate, mammals, non-mammalian eukaryotes, and the like. The present disclosure is not limited by the source of the tissue sample.

In some embodiments, the biological sample as described herein can be stained or imaged using any of the exemplary methods described herein.

In some embodiments of any of the methods described herein, the target analyte can be a protein or a nucleic acid. Non-limiting examples of a target nucleic acid include DNA analytes such as genomic DNA, methylated DNA, specific methylated DNA sequences, fragmented DNA, mitochondrial DNA, in situ synthesized PCR products, and RNA/DNA hybrids.

Non-limiting examples of the target nucleic acid also include RNA analytes such as various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g. present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), micro RNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments, the target nucleic acid can include a mutation (e.g., a disease-causing mutation, e.g., a cancer-causing mutation). In some embodiments, the target nucleic acid includes single nucleotide polymorphism, a gene amplification, insertions, deletions, or a chromosomal translocation.

An analyte capture agent of the plurality of analyte capture agents comprises an analyte binding moiety barcode, an analyte capture sequence, and an analyte binding moiety (e.g., any of the analyte binding moieties described herein) that specifically binds to a target analyte (e.g., any of the target analytes described herein).

In some embodiments of any of the methods described herein, a probe containing an analyte capture domain can be extended using the target analyte specifically bound to the capture domain as a template.

1. A composition, comprising:
a nucleic acid array comprising a support having a plurality of first probes affixed to the support, wherein each of the first probes comprises 2, 3, 4, or 5 copies of a first nucleotide hybridization sequence; and
a plurality of second probes, wherein each of the second probes comprises a second hybridization sequence complementary to the first nucleotide hybridization sequence;
wherein:
two or more of the copies of the first nucleotide hybridization sequence in each of the first probes are hybridized to the second nucleotide hybridization sequence of the second probes;
each of the first probes comprises a unique spatial barcode associated with a unique location on the nucleic acid array; and
the unique spatial barcode of each of the first probes does not substantially hybridize to a nucleic acid in a biological sample.

2. The composition of claim 1, wherein each of the second probes includes 2, 3, 4, or 5 copies of a third nucleotide hybridization sequence, and the composition additionally comprises:
a plurality of third probes, wherein each of the third probes comprises a fourth nucleotide hybridization sequence complementary to the third nucleotide hybridization sequence;
wherein two or more of the copies of the third nucleotide hybridization sequence in each of the second probes are hybridized to the fourth nucleotide hybridization sequence of the third probes.

3. The composition of claim 2, wherein a melting temperature for a duplex formed by hybridization of a copy of the first nucleotide hybridization sequence with a copy of the second nucleotide hybridization sequence is greater than a melting temperature for a duplex formed by hybridization of a copy of the third nucleotide hybridization sequence with a copy of the fourth nucleotide hybridization sequence.

4. The composition of claim 1, wherein each of the first probes comprises 2 copies of the first nucleotide hybridization sequence.

5. The composition of claim 1, wherein each of the first probes further comprises a capture domain.

6. The composition of claim 5, wherein the capture domain comprises a poly(T) nucleotide sequence.

7. The composition of claim 4, wherein each of the second probes comprises 2 copies of the second nucleotide hybridization sequence.

8. The composition of claim 5, wherein the capture domain is bound to an analyte or analyte capture agent from a biological sample.

9. The composition of claim 1, wherein each of the first probes further comprises a unique molecular identifier.

10. The composition of claim 1, wherein each of the second probes further comprises a unique molecular identifier.

11. The composition of claim 1, wherein each of the second probes further comprises a capture domain.

12. The composition of claim 11, wherein the capture domain comprises a poly(T) nucleotide sequence.

13. The composition of claim 11, wherein the capture domain is bound to an analyte or analyte capture agent from a biological sample.

14. The composition of claim 2, wherein each of the third probes comprises a unique spatial barcode associated with a unique location on the nucleic acid array.

15. The composition of claim 2, wherein each of the third probes further comprises a capture domain.

16. The composition of claim 15, wherein the capture domain comprises a poly(T) nucleotide sequence.

17. The composition of claim 15, wherein the capture domain is bound to an analyte or analyte capture agent from a biological sample.

18. The composition of claim 1, wherein the composition further comprises a biological sample.

19. The composition of claim 1, wherein the composition further comprises a biological sample overlaid onto the support of the nucleic acid array.

20. The composition of claim 18, wherein the biological sample is a tissue section.

21. The composition of claim 20, wherein the tissue section is a fixed tissue section.

22. The composition of claim 20, wherein the tissue section is a fresh frozen tissue section.
